(19) United States
(12) Patent Application Publication
(10) Pub. No.: US 2008/0281385 A1
(43) Pub. Date: Nov. 13, 2008

(54) System and Method For Phototherapy With Semiconductor Light-Emitting Element

(76) Inventors: Shunko Albano Inada, Yokkaichi-shi (JP); Hiroshi Amano, Nagoya-shi (JP); Akimichi Morita, Nagoya-shi (JP)

Correspondence Address: OLIFF & BERRIDGE, PLC, P.O. BOX 320850, ALEXANDRIA, VA, 22320-4850, US

(21) Appl. No.: 12/086,016

(22) PCT Filed: Dec. 5, 2006
(86) PCT No.: PCT/JP2006/324258

§ 371 Date: Jun. 16, 2008

(30) Foreign Application Priority Data
Dec. 5, 2005 (JP) 2005-350705

(51) Int. Cl. A61B 18/18

(52) U.S. Cl. 607/94

(57) ABSTRACT

A system for phototherapy and a method for phototherapy are disclosed. The system redeems the faults of conventional fluorescent bulb-type phototherapeutic system. Moreover, the system is small size, lightweight and thus portable, and the system renders a topical irradiation for only a diseased portion of skin possible in combination with an irradiation control system. A semiconductor ultraviolet ray light-emitting element is prepared, and a given ultraviolet ray is generated from this semiconductor ultraviolet ray light-emitting element, and the diseased site is irradiated in order to treat said diseased site.

### TECHNICAL FIELD

The invention relates to a method of phototherapy by a semiconductor light-emitting element, and a system of phototherapy with a semiconductor light-emitting element.

### BACKGROUND ART

The phototherapy which belongs to a technical field of this invention has long history, and it is known that Hippocrates used the heliotherapy for prevention of dermatosis around B.C. 460 in ancient times. Neels FINZEN of Denmark used an artificial light first against the treatment using sunrays, i.e., a natural light. Carbon arc lamp was contrived for the first time in 1893, and the remarkable curative effect was confirmed for lupus vulgaris.

In Japan, it is said that the phototherapy with carbon arc lamp is used for the first time in the Tokyo University, department of dermatology in 1903. Although every initial apparatus was articles imported, the Japan-made carbon arc lamp is developed by the technical cooperation of the Yoshimasa UTSUNOMIYA and IBIDEN CO., LTD. in 1932 (Showa 7).

As for the artificial light source, including a carbon arc lamp in the phototherapy that had been taken for a long time, a close approximation to the sun light spectrum is used. Moreover, above all, in late years the fluorescent bulb which has middle wavelength ultraviolet rays (UV-B) or long wavelength ultraviolet rays (UV-A) in the wavelength area came to be used, and the ultraviolet rays treatment was generalized as a treatment of the disease of skin. However, it is recognized that it is necessary for treatment to use the rays of a particular wavelength each for various skin diseases as the mechanism of phototherapy is elucidated. The recognition is based on the grounds of that the objective is to minimize a side effect and to maximize an effect.

For example, as one of the hospital which performs phototherapy from the earliest time in the world, the Nagoya City University hospital carries out a treatment of psoriasis by the UV-B wave with a wavelength of 311-313 nm. As for the light source, the fluorescence bulb is used, which Philips Corporation of the Netherlands developed. The phototherapy system using the fluorescence bulb is characterized in that the system makes a large area irradiation possible and the spectral line width of very narrow ultraviolet light is obtained except that it is limited to only 311-313 nm.

### DISCLOSURE OF INVENTION

### Technical Problem

However, there are the following problems: (1) the equipment is large-scale and not portable; (2) a big area is required for installation; (3) a normal site is irradiated owing to a large area irradiation; (4) there is a possibility that the medical worker is exposed to irradiation; and (5) the selectivity of wavelength is poor because an available wavelength is restricted by the fluorescence bulb (Today, the ultraviolet light with very narrow spectral line width is only 311-313 nm), and the like. Accordingly, the spread was prohibited. It is an object of this invention to provide a system for phototherapy and a method for phototherapy. The system redeems a fault of the conventional fluorescent bulb-type phototherapeutic system. Moreover, the system is small size, lightweight and thus portable, and the system renders a topical irradiation for only a diseased portion of skin possible in combination with an irradiation control system.

### Solution to Problem

In order to attain the above-mentioned object, this invention relates to a system of phototherapy with a semiconductor light-emitting element, comprising;

a semiconductor ultraviolet ray light-emitting element for generating a given ultraviolet ray,
wherein the system is constructed so as to irradiate a diseased site with the ray to treat the diseased site.

In addition, this invention relates to a method of phototherapy with a semiconductor light-emitting element, comprising the steps of: preparing a semiconductor ultraviolet ray light-emitting element, and generating a given ultraviolet ray from the semiconductor ultraviolet ray light-emitting element and irradiating a diseased site with the ray to treat the diseased site.

The inventors have come to develop the semiconductor ultraviolet rays light-emitting element which can make the high-intensity ultraviolet rays generation and emission highly effective in the process of research and development of a semiconductor light-emitting element over many years. This semiconductor ultraviolet rays light-emitting element has the following features.

1. Comparing with the conventional glass tube, it is a) microminiature, b) lightweight, c) point light source, and d) the combination of various wavelengths is possible, e) the intensity variability is easy. Furthermore, f) the emission wavelength range is narrow and it is possible to emit light selectively only in a specific wavelength. 2. The miniaturization of device is easy. 3. The irradiation is possible as a topical method (target type irradiation, and spot delivery). As a result, the irradiation is not performed in a normal site without a lesion, and therefore it is possible to reduce the side effects of irradiation in the normal site. In addition, it is possible to avoid the ultraviolet rays exposure to a medical worker (on the part of operator which irradiates).

Therefore, the above-mentioned semiconductor ultraviolet rays light-emitting element has many advantages which resolve the various faults of the conventional fluorescence bulb as mentioned above. As a result, the semiconductor ultraviolet rays light-emitting element which has such a feature is used instead of the fluorescence bulb mentioned above in the conventional phototherapy, most of the faults based on the fluorescence bulb type phototherapy which was mentioned above can be resolved.

Moreover, since an alternative ultraviolet rays wavelength can be used by means of the semiconductor ultraviolet-rays light-emitting element, the additional action and effect can be obtained so that the side effects, such as an erythema reaction, pigmentation, carcinogenicity and the like are reduced to the minimum. Furthermore, since a semiconductor ultraviolet rays light-emitting element is a point light source, the light of uniform intensity can be irradiated to a diseased site. Moreover, since it is small size and lightweight, the position to a diseased site can be determined easily and the distance from the diseased site can be determined easily.

Furthermore, in a mode of the invention, the semiconductor ultraviolet rays light-emitting element can be used to operate the ultraviolet ray on the diseased site. Since it is difficult that the spot of the ultraviolet rays emitted from the semiconductor ultraviolet rays light-emitting element is set always to meet with the size of a diseased site, such a operation can make the irradiation of intended ultraviolet rays possible over the whole diseased site.

Moreover, in another mode of the invention, the semiconductor ultraviolet ray light-emitting element can compose a plurality of semiconductor ultraviolet ray light-emitting elements, wherein these semiconductor ultraviolet ray light-emitting elements are arranged in a shape of array, a portion of the plurality of semiconductor ultraviolet ray light-emitting elements corresponding to the diseased site is turn on, the ultraviolet ray is irradiated over the whole diseased site. As mentioned above, it is difficult that the spot of the ultraviolet rays emitted from the semiconductor ultraviolet rays light-emitting element is set always to meet with the size of a diseased site. Therefore, the plurality of semiconductor ultraviolet-rays light-emitting elements are arranged in a shape of an array, and only the predetermined portion of light-emitting element is turn on, and thereby the intended ultraviolet rays can be irradiated over the whole diseased site.

Furthermore, in still another mode of the invention, a means for imaging a subject to be irradiated with the ultraviolet ray can be composed, wherein the subject to be irradiated with the ultraviolet ray is imaged to obtain a given imaging data and thereafter the diseased site is specified based on this imaging data.

In addition, the method and device of the invention can be used in any disease, especially preferably in a skin disease. Specifically, the disease can be at least one selected from the group consisting of an intractable eczema, a dyshidrotic eczema, a cutaneous T cell lymphoma, an atopic darmatitis, an alopecia greata, a keloid, a cicatrix, an atrophia cutis linear (a stretch mark), a scleroderma, a leukoplakia, a psoriasis, a palmoplantar pustulosis, a chronic eczema.

As explained above, according to the invention, a system for phototherapy and a method for phototherapy can be provided. The faults of the conventional fluorescence bulb-type phototherapy system can be compensated. Moreover, the system is small size, lightweight and thus portable, and the system renders a topical irradiation for only a diseased portion of skin possible in combination with an irradiation control system.

### BEST MODE FOR CARRYING OUT THE INVENTION

The details and other features and advantages of the invention will be described in detail based on the best mode as follows.

In the invention, firstly, the predetermined semiconductor ultraviolet rays light-emitting element is prepared. This semiconductor ultraviolet rays light-emitting element can be used solely and can be used to arrange a plurality of the elements in the shape of array.

If the single semiconductor ultraviolet rays light-emitting element is used, it is difficult that in the diseased site the spot of the ultraviolet rays emitted from the semiconductor ultraviolet rays light-emitting element is set always to meet with the size of a diseased site. Therefore, the ultraviolet rays are operated so that the intended ultraviolet rays can be irradiated over the whole diseased site.

In addition, if a plurality of semiconductor ultraviolet rays light-emitting elements are arranged in a shape of array, a portion of the plurality of semiconductor ultraviolet ray light-emitting elements corresponding to the diseased site is turn on, the ultraviolet ray is irradiated over the whole diseased site. As mentioned above, it is difficult that the spot of the ultraviolet rays emitted from the semiconductor ultraviolet rays light-emitting element is set always to meet with the size of a diseased site. Therefore, the plurality of semiconductor ultraviolet rays light-emitting elements is arranged in a shape of an array, and only the predetermined portion of light emitting element is turn on so that the intended ultraviolet rays can be irradiated over the whole diseased site.

Furthermore, if the above-mentioned semiconductor ultraviolet rays light-emitting element is used, the peak wavelength exists within at least one extend of a range of from 350 nm to 390 nm, from 305 nm to 315 nm and from 200 nm to 305 nm. In other words, the above-mentioned semiconductor ultraviolet rays light-emitting element has an emitting wavelength within such a wavelength extend and thereby becoming useful for the treatment of above-mentioned diseases, in particular the skin disease.

Specifically, if the peak wavelength of the semiconductor ultraviolet rays light-emitting element is within the extend from 350 nm to 390 nm, the element is effective for the treatment of an intractable eczema, a dyshidrotic eczema, a cutaneous T cell lymphoma, an atopic darmatitis, an alopecia areata, a keloid, a cicatrix, an atrophia cutis linear (a stretch mark), a scleroderma, or the like. In addition, if the peak wavelength of the semiconductor ultraviolet rays light-emitting element is within the extend from 305 nm to 315 nm, the element is effective for the treatment of a leukoplakia, a psoriasis, a palmoplantar pustulosis, a chronic eczema, an atopic darmatitis or the like.

In addition, the exposure dose of ultraviolet rays emitted from the semiconductor light-emitting element is not limited to a specific dose as long as the above-mentioned diseases such as a skin disease can be treated. Preferable dose is 1 mW/cm² or more. Hereby, the above-mentioned skin disease or the like can be treated effectually.

Moreover, the upper limit on intensity of ultraviolet rays is also not limited in particular. 10 W/cm² or less is preferable. If the ultraviolet rays are irradiated over the above-mentioned value, the side effects, such as an erythema reaction, pigmentation, carcinogenicity and the like may be generated so that the curative effect can not be elicited sufficiently.

Furthermore, as the above-mentioned semiconductor ultraviolet rays light-emitting element, any available element can be used. However, under the present conditions, there is almost no practical implementation of semiconductor ultraviolet rays light-emitting element which can make the high-intensity ultraviolet rays generation and emission highly effective. Therefore, it is preferable to use the semiconductor ultraviolet rays light-emitting element developed by the present inventors as explained below in detail.

### BRIEF DESCRIPTION OF THE DRAWINGS

FIG. 1 is a schematic view of constitution showing an example of the semiconductor ultraviolet rays light-emitting element to use in the invention.

FIG. 2 is a schematic view showing an outline of the band structure in the valence band of GaN and AlN which are a group III nitride semiconductor.

FIG. 3 is a similar schematic view showing an outline of the band structure in the valence band of GaN and AlN which are a group III nitride semiconductor.

FIG. 4 is a schematic view of constitution showing an example of the semiconductor ultraviolet rays light-emitting element to use in the invention.

FIG. 5 is a graph showing the emission spectrum of the semiconductor ultraviolet rays light-emitting element which used in an embodiment.

(The First Semiconductor Ultraviolet Rays Light-Emitting Element)

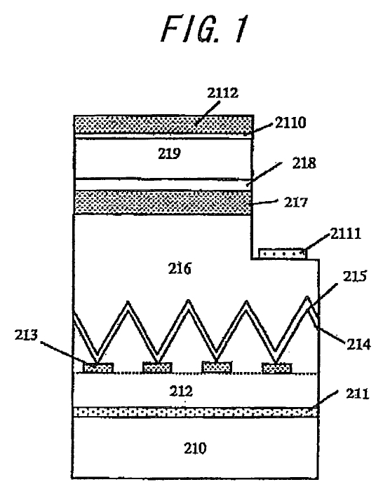

FIG. 1 is a schematic view of constitution showing an example of the semiconductor ultraviolet rays light-emitting element to use in the invention. As shown in FIG. 1, a light-emitting element in this example is an AlGaN based semiconductor light-emitting element. At first, on sapphire substrate 210, AlN layer 211 and AlGaN layer 212 are grown by method of organometallic compounds vapor phase epitaxy, and thereafter SiO₂ mask 213 is formed periodically in the direction of (1-100) on AlGaN layer 212 by an EB vapor deposition device. Subsequently, AlGaN facet layer 214 is formed by method of organic metal vapor phase epitaxy to cover SiO₂ mask 213 completely in order to appear a facet 214 of (11-22).

Subsequently, via AlN layer 215, the planarization is performed by Si-added n-type planarizing layer 216 of Al_{0.50}Ga_{0.50}N showing the n-type conductivity with carrier density of 2×10¹⁸ cm⁻³, and thereafter a multiquantum well structure active layer 217 of Al_{0.17}Ga_{0.83}N/Al_{0.25}Ga_{0.75}N, a p-type blocking layer 218 of Al_{0.60}Ga_{0.40}N with carrier density of 8×10¹⁷ cm⁻³, a p-type cladding layer 219 of Al_{0.50}Ga_{0.50}N with carrier density of 1×10¹⁸ cm⁻³ and a p-type contact layer 2110 of GaN with carrier density of 1×10¹⁸ cm⁻³ are laminated in order of precedence, and a n-type electrode 2111 comprising of Ti/Al and a p-type electrode 2112 comprising of Ni/Au are formed and then an AlGaN based semiconductor light-emitting element (a diode) is manufactured.

As for the crystallinity of AlGaN realized by this crystal growth, the dislocation-density is as low as 1×10⁸ cm⁻², and for the p-type blocking layer and p-type cladding layer, a AlGaN is used in AlN molar fraction of 0.6 and 0.5, respectively. Consequently, the p-type blocking layer and the p-type cladding layer will compose of a p-type AlGaN layer of AlN molar fraction of more than 0.15 with a wide gap and a high carrier density of 1×10¹⁸ cm⁻³.

The semiconductor ultraviolet rays light-emitting element obtained after this manner shows such an emission property having a peak at 313 nm.

Furthermore, in this example, the p-type blocking layer and the p-type cladding layer have the carrier density of 1×10¹⁸ cm⁻³, but the element can generate the ultraviolet rays of sufficient intensity and can emit the light if the requirement of 1×10¹⁶ cm⁻³ or more is satisfied. Similarly, the p-type blocking layer and the p-type cladding layer have the AlN molar fraction of 0.6 and 0.5, respectively, but the element can generate the ultraviolet rays of sufficient intensity and can emit the light if the molar fraction is 0.15 or more.

In addition, the p-type blocking layer and the p-type cladding layer have preferably a half bandwidth of 800 seconds or less on X-ray rocking curve of (0002) diffraction, and have preferably a half bandwidth of 1000 seconds or less on X-ray rocking curve of (10-10) diffraction. Hereby, the crystal quality of these layers is improved significantly and thereby the intended high-efficiency ultraviolet rays can be generated and can be emitted.

Moreover, in this example, the p-type AlGaN blocking layer 218 having AlN molar fraction of 0.6 with carrier density of 8×10¹⁷ cm⁻³ and the p-type AlGaN cladding layer 219 having AlN molar fraction of 0.5 with carrier density of 1×10¹⁸ cm⁻³ are used, but these property values can be properly changed within the scope of the invention in the AlGaN based semiconductor light-emitting element shown in FIG. 1 according to the desired emission wavelength.

Furthermore, as mentioned above, the p-type blocking layer and the p-type cladding layer is composed of the p-type AlGaN layer having the carrier density of more than 5×10¹⁷ cm⁻³ and the AlN molar fraction of more than 0.3. Such a p-type AlGaN layer shows the following properties.

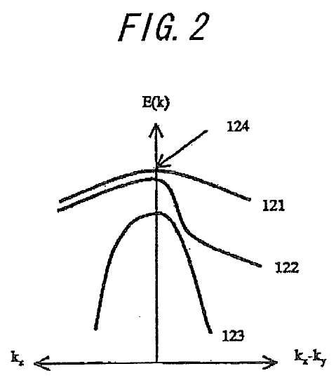

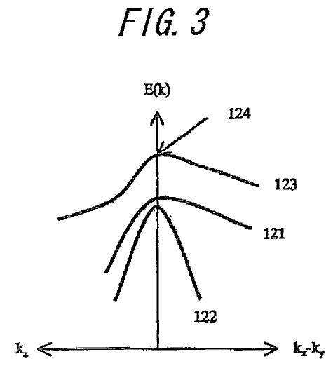

The FIGS. 2 and 3 are a schematic view showing an outline of the band structure in the valence band of GaN and AlN which are a group III nitride semiconductor. The band structure in the group III nitride semiconductor is divided to three of a heavy hole (HH) 121, a light hole (LH) 122, and a crystal field splitting hole (CH) 123. In addition, in GaN and AlN, a band of the top at Γ point 124 is HH and CH, respectively and it is a characteristic that these differ each other.

In AlGaN, when an AlN molar fraction is low, the HH and LH are higher than the CH, but as the AlN molar fraction is increased, the CH rises relatively compared with HH and LH, and then these three bands are almost piled up at AlN molar fraction of around 0.40. In addition, if the AlN molar fraction is increased further, the CH is higher than the HH and LH. Based on such properties, if the AlN molar fraction is of from 0 to 0.3, the increase of state density enables the carrier density to decrease, but if the AlN molar fraction is of more than 0.3, the carrier density is increased and the maximum value is taken around 0.4, and thereby the AlN becomes also p-type conductive. Therefore, mainly, originating in this kind of p-type AlGaN, the semiconductor ultraviolet ray light-emitting element of this example can make the high-intensity ultraviolet rays emission highly effective.

(The Second Semiconductor Ultraviolet Rays Light-Emitting Element)

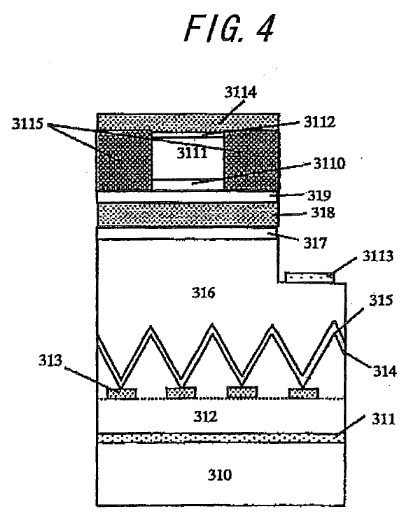

FIG. 4 is a schematic view of constitution showing an example of the semiconductor ultraviolet rays light-emitting element to use in the invention. As shown in FIG. 4, a light-emitting element in this example is also an AlGaN based semiconductor light-emitting element. At first, on sapphire substrate 310, AlN layer 311 and AlGaN layer 312 are grown by method of organometallic compounds vapor phase epitaxy, and then SiO₂ mask 313 is formed periodically in the direction of [1-100] on AlGaN layer 312 by an EB vapor deposition device. Subsequently, AlGaN facet layer 314 is formed by method of organic metal vapor phase epitaxy to appear a AlGaN facet 314 of (11-22) in order to cover SiO₂ mask completely.

Thereafter, via AlN layer 315, the planarization is performed by Si-added n-type planarizing layer 316 of Al_{0.50}Ga_{0.50}N showing the n-type conductivity with carrier density of 2×10¹⁸ cm⁻³, and then a guide layer 317 of undoped Al_{0.38}Ga_{0.62}N, a multiquantum well structure active layer 318 of Al_{0.17}Ga_{0.83}N/Al_{0.25}Ga_{0.75}N, a guide layer 319 of undoped Al_{0.38}Ga_{0.62}N, a p-type blocking layer 3110 of Al_{0.60}Ga_{0.40}N with carrier density of 8×10¹⁷ cm⁻³, a p-type cladding layer 3111 of Al_{0.50}Ga_{0.50}N with carrier density of 1×10¹⁸ cm⁻³ and a p-type contact layer 3112 of GaN with carrier density of 1×10¹⁸ cm⁻³ are laminated. Subsequently, a n-electrode 3113 comprising of Ti/Al, a p-electrode 3114 comprising of Ni/Au, an electric current structure layer comprising of SiO₂ and the like are formed. Consequently, the illustrated AlGaN based semiconductor light-emitting element started functioning as a laser diode of ridge type.

As for the crystallinity of AlGaN realized by this crystal growth, the dislocation density is as low as 1×10⁸ cm⁻², and for the p-type blocking layer and p-type cladding layer, a AlGaN is used in AlN molar fraction of 0.6 and 0.5, respectively, a wide gap with AlN molar fraction of more than 0.3 and a high carrier density of 8×10¹⁷ cm⁻³ and 1×10¹⁸ cm⁻³ can be realized.

The semiconductor ultraviolet rays light-emitting element obtained after this manner shows such an emission property having a peak at 313 nm.

Furthermore, in this example, the p-type blocking layer and the p-type cladding layer have the carrier density of 1×10¹⁸ cm⁻³, but the element can generate the ultraviolet rays of sufficient intensity and can emit the light if the requirement of 1×10¹⁶ cm⁻³ or more is satisfied. Similarly, the p-type blocking layer and the p-type cladding layer have the AlN molar fraction of 0.6 and 0.5, respectively, but the element can generate the ultraviolet rays of sufficient intensity and can emit the light if the molar fraction is 0.15 or more.

In addition, the p-type blocking layer and the p-type cladding layer have preferably a half bandwidth of 800 seconds or less on X-ray rocking curve of (0002) diffraction, and have preferably a half bandwidth of 1000 seconds or less on X-ray rocking curve of (10-10) diffraction. Hereby, the crystal quality of these layers is improved significantly and thereby the intended high-efficiency ultraviolet rays can be generated and can be emitted.

Moreover, in this example, the p-type AlGaN blocking layer 3110 having AlN molar fraction of 0.6 with carrier density of 8×10¹⁷ cm⁻³ and the p-type AlGaN cladding layer 3111 having AlN molar fraction of 0.5 with carrier density of 1×10¹⁸ cm⁻³ are used, but these property values can be properly changed within the scope of the invention in the AlGaN based semiconductor light-emitting element shown in FIG. 4 according to the desired emission wavelength.

Furthermore, as for the p-type AlGaN layer used in this example, the properties shown in FIGS. 2 and 3 are shown.

### EXAMPLES

First, in order to verify that the semiconductor ultraviolet ray light-emitting element is useful for phototherapy, the comparison was performed as to tumor cell death necessary for phototherapy. In the comparison, the conventional broadband UVA irradiation device of partial body UVA1 (340-400 nm) irradiation Sellamed system and the light emitting diode which was developed at this time comprising the group III nitride semiconductor with a peak wavelength of 365 nm shown in FIG. 1 are used. The irradiation is performed in the same energy and the size of LED is 0.5 mm×0.5 mm.

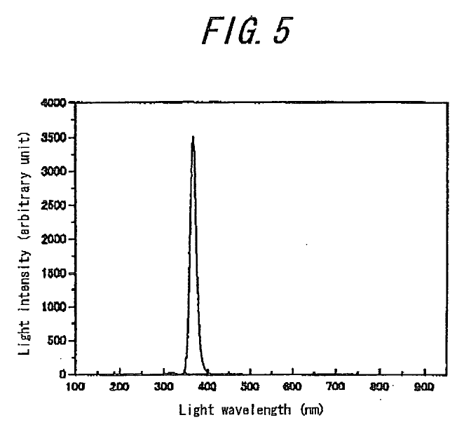

FIG. 5 shows the emission spectrum of the ultraviolet rays LED which used at this time. In Tables 1 and 2, the irradiation intensity of 80 mW/cm², the changes of exposure dose of 10, 20, 30 J/cm², and the ratio of apoptosis caused in tumor cells (Table 1), and the ratio of necrosis (Table 2) by ultraviolet rays irradiation with two light source devices are summarized.

TABLE 1

| | UVA1 | LED | | |
|---|---|---|---|---|
| | Ratio of | Ratio of | UVA | LED |
| | Apoptosis[%] | Apoptosis[%] | Average | Average |
| Control | 19.7 | 19.7 | 19.86 | 19.86 |
| | 20 | 20 | | |
| | 19.87 | 19.87 | | |
| (10)J/cm² | 22.11 | 23.97 | 22.89 | 21.68 |
| | 24.17 | 20.32 | | |
| | 22.4 | 20.74 | | |
| (20)J/cm² | 29.41 | 28.99 | 30.11 | 27.28 |
| | 26.64 | 22.75 | | |
| | 34.29 | 30.09 | | |
| (30)J/cm² | 49.36 | 37.54 | 52.82 | 43.59 |
| | 56.83 | 47.16 | | |
| | 52.28 | 46.06 | | |

TABLE 2

| | | | |
|---|---|---|---|
| UVA1 | LED | | |
| Ratio of Necrosis | Ratio of Necrosis | UVA | LED |
| [%] | [%] | Average | Average |

| | | | | |
|---|---|---|---|---|
| Control | 20.98 | 20.98 | 20.72 | 20.71 |
| | 19.51 | 19.51 | | |
| | 21.68 | 21.63 | | |
| (10)J/cm² | 24 | 19.68 | 22.08 | 18.47 |
| | 23.95 | 17.52 | | |
| | 18.28 | 18.22 | | |
| (20)J/cm² | 25.57 | 25.74 | 25.90 | 24.72 |
| | 22.35 | 20.77 | | |
| | 29.77 | 27.65 | | |
| (30)J/cm² | 38.28 | 34.61 | 41.87 | 38.37 |
| | 45.61 | 40.35 | | |
| | 41.72 | 40.15 | | |

As is clear from these Tables, LED irradiation device which was developed at this time showed the effect equivalent to the conventional lamp-type broadband UVA irradiation device.

Next, the LEDs of 0.5 mm×0.5 mm/one chip were arranged to form the array shape of 20 chips×20 chips, total 400 chips, the area of 20 cm×20 cm. The portion of LEDs was partially lighted, and the above-mentioned irradiation was performed, upon which the apoptosis and necrosis were observed in only the lightning portion of LEDs.

Based on this result, for a patient suffer from an atopic dermatitis, (1) the dermatitis-bearing site was photographed by a digital camera, (2) the obtained photography data was analyzed to prepare a dermatitis map data, (3) a prototype LED a array device was lighted against the site of dermatitis to continue such a treatment of irradiation for 125 seconds, upon which a curative effect was observed.

If energy was the same, it was confirmed that an approximately equal effect was obtained by using LEDs with peak wavelength between from 350 nm to 390 nm in this experimentation.

Besides, the effects for various dermatitides were observed in the wavelength regions of from 305 nm to 315 nm, from 315 nm to 350 nm, from 200 nm to 305 nm, respectively. In addition, for various skin diseases, the curative effects were partially observed in a combination of UV-LED and visible LED, and in a combination of UV-LED and infrared rays LED, or a combination of UV-LED, visible LED and infrared rays LED.

As above, the invention has been described in detail on the basis of the forms of the embodiment while listing the concrete examples, but it should be construed that the invention is not limited to the above-mentioned contents and that every variation or change are possible insofar as the scope of invention does not deviate.

### INDUSTRIAL APPLICABILITY

As mentioned above, according to the invention, a system for phototherapy and a method for phototherapy can be provided. The system redeems the faults of conventional fluorescent bulb-type phototherapeutic system. Moreover, the system is small size, lightweight and thus portable, and the system renders a topical irradiation for only a diseased portion of skin possible in combination with an irradiation control system.

1. A method of phototherapy with a semiconductor light-emitting element, comprising the steps of:
preparing a semiconductor ultraviolet ray light-emitting element, and generating a given ultraviolet ray from the semiconductor ultraviolet ray light-emitting element in order to irradiate a diseased site with the given ultraviolet ray to treat the diseased site.

2. A method of phototherapy with a semiconductor light-emitting element according to claim 1, wherein the semiconductor ultraviolet ray light-emitting element is used to operate the ultraviolet ray on the diseased site.

3. A method of phototherapy with a semiconductor light-emitting element according to claim 1, wherein the semiconductor ultraviolet ray light-emitting element composes a plurality of semiconductor ultraviolet ray light-emitting elements, these semiconductor ultraviolet ray light-emitting elements are arranged in a shape of an array, a portion of the plurality of semiconductor ultraviolet ray light-emitting elements corresponding to the diseased site are turned on, the ultraviolet ray is irradiated over the whole diseased site.

4. A method of phototherapy with a semiconductor light-emitting element according to claim 1, wherein the semiconductor ultraviolet ray light-emitting element has a peak wavelength within at least one of a range of from 350 nm to 390 nm, 305 nm to 315 nm, and 200 nm to 305 nm.

5. A method of phototherapy with a semiconductor light-emitting element according to claim 1, wherein an ultraviolet ray exposure dose with the semiconductor ultraviolet ray light-emitting element is 1 mW/cm² or more.

6. A method of phototherapy with a semiconductor light-emitting element according to claim 1, wherein the ultraviolet ray exposure dose with the semiconductor ultraviolet ray light-emitting element is 10 mW/cm² or less.

7. A method of phototherapy with a semiconductor light-emitting element according claim 1, wherein the diseased site is a diseased site of skin.

8. A method of phototherapy with a semiconductor light-emitting element according to claim 7, wherein the diseased site is at least one site selected from a group consisting of an intractable eczema, a dyshidrotic eczema, a cutaneous T cell lymphoma, an atopic darmatitis, an alopecia areata, a keloid, a cicatrix, an atrophia cutis linear (a strech mark), a scleroderma, a leukoplakia, a psoriasis, a palmoplantar pustulosis, and a chronic eczema.

9. A method of phototherapy with a semiconductor light-emitting element according to claim 1, wherein a subject to be irradiated with the ultraviolet ray is imaged to obtain given imaging data and thereafter the diseased site is specified based on this imaging data.

10. A method of phototherapy with a semiconductor light-emitting element according to claim 1, wherein the semiconductor ultraviolet ray light-emitting element is an AlGaN-based semiconductor light-emitting element having a carrier density of more than 5×10¹⁷ cm⁻³, and the element comprises a p-type AlGaN layer specified by an AlN molar fraction of more than 0.15 as at least one of a p-type AlGaN cladding layer and a p-type AlGaN blocking layer.

11. A method of phototherapy with a semiconductor light-emitting element according to claim 10, wherein the semiconductor ultraviolet ray light-emitting element comprises an AlN layer, an AlGaN layer, an AlN layer formed on an AlGaN(11-22) facet, a n-type AlGaN planarizing layer, an AlGaN active layer, the p-type AlGaN blocking layer, and the p-type AlGaN cladding layer in order of precedence on a given substrate.

12. A method of phototherapy with a semiconductor light-emitting element according to claim 10, wherein the semiconductor ultraviolet ray light-emitting element comprises an AlN layer, an AlGaN layer, an AlN layer formed on an AlGaN (11-22) facet, a n-type AlGaN planarizing layer, a first AlGaN guide layer, an AlGaN active layer, a second AlGaN guide layer, the p-type AlGaN blocking layer, and the p-type AlGaN cladding layer in order of precedence on a given substrate.

13. A method of phototherapy with a semiconductor light-emitting element according to claim 12, wherein the semiconductor ultraviolet ray light-emitting element has an electric current structure layer formed adjacent to the p-type AlGaN blocking layer and the p-type AlGaN cladding layer, and the element presents a ridge type.

14. A method of phototherapy with a semiconductor light-emitting element according to claim 10, wherein the p-type AlGaN layer has a half bandwidth of 800 seconds or less on X-ray rocking curve of (0002) diffraction.

15. A method of phototherapy with a semiconductor light-emitting element according to claim 10, wherein the p-type AlGaN layer has a half bandwidth of 1000 seconds or less on X-ray rocking curve of (10-10) diffraction.

16. A method of phototherapy with a semiconductor light-emitting element according to claim 10, wherein the p-type AlGaN layer has a dislocation density of 5×10⁹ cm⁻² or less.

17. A method of phototherapy with a semiconductor light-emitting element according to claim 10, wherein the p-type AlGaN layer satisfies a relation of CH HH, LH, wherein HH is a band zone energy of heavy hole, LH is a band zone energy of light hole, and CH is a band zone energy of crystal field splitting hole.

18. A method of phototherapy with semiconductor light-emitting element according to claim 1, wherein the p-type AlGaN layer has a carrier density of 1×10⁸ cm⁻³ or more.

19. A method of phototherapy with a semiconductor light-emitting element according to claim 1, wherein a subject having the diseased site to be treated with the ultraviolet ray is an animal other than a human being.

20. A system of phototherapy with a semiconductor light-emitting element, comprising:
a semiconductor ultraviolet ray light-emitting element for generating a given ultraviolet ray,
wherein the system is constructed so as to irradiate a diseased site with the ray to treat the diseased site.

21. A system of phototherapy with a semiconductor light-emitting element according to claim 20, wherein the semiconductor ultraviolet ray light-emitting element is used to operate the ultraviolet ray on the diseased site.

22. A system of phototherapy with a semiconductor light-emitting element according to claim 20, wherein the semiconductor ultraviolet ray light-emitting element composes a plurality of semiconductor ultraviolet ray light-emitting elements, these semiconductor ultraviolet ray light-emitting elements are arranged in a shape of an array, a portion of the plurality of semiconductor ultraviolet ray light-emitting elements corresponding to the diseased site is are turned on, the ultraviolet ray is irradiated over the whole diseased site.

23. A system of phototherapy with a semiconductor light-emitting element according to claim 20, wherein the semiconductor ultraviolet ray light-emitting element has a peak wavelength within at least one extend of a range of from 350 nm to 390 nm, 305 nm to 315 nm, and 200 nm to 305 nm.

24. A system of phototherapy with a semiconductor light-emitting element according to claim 20, wherein an ultraviolet ray exposure dose with the semiconductor ultraviolet ray light-emitting element is 1 mW/cm² or more.

25. A system of phototherapy with a semiconductor light-emitting element according to claim 20, wherein the ultraviolet ray exposure dose with the semiconductor ultraviolet ray light-emitting element is 10 mW/cm² or less.

26. A system of phototherapy with a semiconductor light-emitting element according to claim 20, wherein the diseased site is a diseased site of skin.

27. A system of phototherapy with a semiconductor light-emitting element according to claim 26, wherein the diseased site is at least one site selected from the group consisting of an intractable eczema, a dyshidrotic eczema, a cutaneous T cell lymphoma, an atopic darmatitis, an alopecia areata, a keloid, a cicatrix, an atrophia cutis linear (a strech mark), a scleroderma, a leukoplakia, a psoriasis, a palmoplantar pustulosis, and a chronic eczema.

28. A system of phototherapy with a semiconductor light-emitting element according to claim 20, comprising a means for imaging a subject to be irradiated with the ultraviolet ray, wherein the subject to be irradiated with the ultraviolet ray is imaged to obtain a given imaging data and thereafter the diseased site is specified based on this imaging data.

29. A system of phototherapy with a semiconductor light-emitting element according to claim 20, wherein the semiconductor ultraviolet ray light-emitting element is an AlGaN-based semiconductor light-emitting element having a carrier density of more than 5×10¹⁷ cm⁻³, and the element comprises a p-type AlGaN layer specified by an AlN molar fraction of more than 0.15 as at least one of a p-type AlGaN cladding layer and a p-type AlGaN blocking layer.

30. A system of phototherapy with a semiconductor light-emitting element according to claim 29, wherein the semiconductor ultraviolet ray light-emitting element comprises an AlN layer, an AlGaN layer, an AlN layer formed on an AlGaN (11-22) facet, a n-type AlGaN planarizing layer, an AlGaN active layer, the p-type AlGaN blocking layer, and the p-type AlGaN cladding layer in order of precedence on a given substrate.

31. A method of phototherapy with a semiconductor light-emitting element according to claim 29, wherein the semiconductor ultraviolet ray light-emitting element comprises an AlN layer, an AlGaN layer, an AlN layer formed on an AlGaN (11-22) facet, a n-type AlGaN planarizing layer, a first AlGaN guide layer, an AlGaN active layer, a second AlGaN guide layer, the p-type AlGaN blocking layer, and the p-type AlGaN cladding layer in order of precedence on a given substrate.

32. A system of phototherapy with a semiconductor light-emitting element according to claim 31, wherein the semiconductor ultraviolet ray light-emitting element has an electric current structure layer formed adjacent to the p-type AlGaN blocking layer and the p-type AlGaN cladding layer, and the element presents a ridge type.

33. A system of phototherapy with a semiconductor light-emitting element according to claim 29, wherein the p-type AlGaN layer has a half bandwidth of 800 seconds or less on X-ray rocking curve of (0002) diffraction.

34. A system of phototherapy with a semiconductor light-emitting element according to claim 29, wherein the p-type AlGaN layer has a half bandwidth of 1000 seconds or less on X-ray rocking curve of (10-10) diffraction.

35. A system of phototherapy with a semiconductor light-emitting element according to claim 29, wherein the p-type AlGaN layer has a dislocation density of 5×10⁹ cm⁻² or less.

36. A system of phototherapy with a semiconductor light-emitting element according to claim 29, wherein the p-type AlGaN layer satisfies a relation of CH≧HH, LH, wherein HH is a band zone energy of heavy hole, LH is a band zone energy of light hole, and CH is a band zone energy of crystal field splitting hole.

37. A system of phototherapy with a semiconductor light-emitting element according to claim 29, wherein the p-type AlGaN layer has a carrier density of 1×10⁸ cm⁻³ or more.

38. A system of phototherapy with a semiconductor light-emitting element according to claim 20, wherein a subject having the diseased site to be treated with the ultraviolet ray is an animal other than a human being.
